(19) United States
(12) Patent Application Publication
(10) Pub. No.: US 2019/0290929 A1
(43) Pub. Date: Sep. 26, 2019

(54) WEARABLE APPARATUS, METHOD FOR MANUFACTURING THE SAME, AND WEARABLE DEVICE

(71) Applicant: BOE TECHNOLOGY GROUP CO., LTD., Beijing (CN)

(72) Inventor: Zhiqiang Jiao, Beijing (CN)

(21) Appl. No.: 16/316,978

(22) PCT Filed: Feb. 14, 2018
(86) PCT No.: PCT/CN2018/076819

§ 371 Date: Jan. 10, 2019

(30) Foreign Application Priority Data
May 10, 2017 (CN) 201720516876.0

(51) Int. Cl. A61N 5/06; H01L 51/56; H01L 51/52; H01L 51/50; H01L 51/00

(52) U.S. Cl. CPC A61N 5/0621; H01L 51/56; H01L 51/5237; H01L 51/504; H01L 51/0097; A61N 2005/0626; H01L 2251/5338; A61N 2005/0663; A61N 2005/0659; A61N 2005/0653; A61N 2005/0645; A61N 5/0613

(57) ABSTRACT

The embodiments of the present application disclose a wearable apparatus, a method for manufacturing the same, and a wearable device. The wearable apparatus may include a flexible Organic Light Emitting Diode (OLED) device including a first light-emitting layer having a first peak emission wavelength and a second light-emitting layer stacked on the first light-emitting layer, wherein the second light-emitting layer has a second peak emission wavelength which is different from the first peak emission wavelength; and a controller connected to the flexible OLED device to control an operation of the flexible OLED device.

### CROSS-REFERENCE TO RELATED APPLICATION(S)

This application is the national phase of PCT Application No. PCT/CN2018/076819 filed on Feb. 14, 2018, which in turn claims priority to the Chinese Patent Application No. CN201720516876.0, filed on May 10, 2017, which is incorporated herein by reference in its entirety.

### TECHNICAL FIELD

Embodiments of the present disclosure relate to the field of organic light-emission, and more particularly, to a wearable apparatus having an organic light-emitting device, a method for manufacturing the same, and a wearable device.

### BACKGROUND

Generally, for a subject suffering from certain diseases, it is possible to help healing of the diseases by illuminating the subject with light of different wavelengths. However, existing light illumination devices are complicated to operate and have a poor user experience.

Therefore, there is a need for a technical solution to eliminate or alleviate the above technical problems.

### SUMMARY

At least one embodiment of the present disclosure provides a wearable apparatus, a method for manufacturing the same, and a wearable device, which at least partially solve the above technical problems.

According to an aspect of the present disclosure, there is proposed a wearable apparatus, comprising:

a flexible Organic Light Emitting Diode (OLED) device comprising a first light-emitting layer having a first peak emission wavelength and a second light-emitting layer stacked on the first light-emitting layer, wherein the second light-emitting layer has a second peak emission wavelength which is different from the first peak emission wavelength; and

a controller connected to the flexible OLED device to control an operation of the flexible OLED device.

According to an exemplary embodiment, the peak emission wavelength of the first light-emitting layer is in a range of 380 nm to 500 nm or 650 nm to 1000 nm.

According to an exemplary embodiment, the peak emission wavelength of the first light-emitting layer is in one of a range of 380 nm to 500 nm and a range of 650 nm to 1000 nm, and the peak emission wavelength of the second light-emitting layer is in the other of the range of 380 nm to 500 nm and the range of 650 nm to 1000 nm.

According to an exemplary embodiment, the wearable apparatus further comprises: a light transmittable liner disposed on a light exiting side of the flexible OLED device.

According to an exemplary embodiment, the light transmittable liner comprises at least one removable fabric layer.

According to an exemplary embodiment, the at least one removable fabric layer comprises a plurality of removable fabric layers which have meshes.

According to an exemplary embodiment, the wearable apparatus further comprises: a light shielding layer which covers one side of the flexible OLED device opposite to the light exiting side to shield light emitted by the flexible OLED device to an external environment.

According to an exemplary embodiment, the wearable apparatus further comprises: a temperature sensor configured to detect a temperature on a light exiting side of the flexible OLED device, wherein the temperature sensor is connected to the controller so that the controller controls to turn off the flexible OLED device when the detected temperature is greater than a temperature threshold.

According to an exemplary embodiment, the wearable apparatus further comprises: a timer configured to count turn-on time of the flexible OLED device so that the flexible OLED device is turned off when the counted turn-on time is greater than a time threshold.

According to another aspect of the present disclosure, there is provided a wearable device, comprising at least one wearable apparatus according to the embodiments of the present disclosure.

According to an exemplary embodiment, the at least one wearable apparatus comprises a plurality of wearable apparatuses, wherein at least some of the plurality of wearable apparatuses is connected to each other via length adjustable mechanisms.

According to yet another aspect of the present disclosure, there is provided a method for manufacturing a wearable apparatus, comprising: forming a flexible Organic Light Emitting Diode (OLED) device on a substrate, wherein the flexible OLED device comprises a first light-emitting layer having a first peak emission wavelength and a second light-emitting layer stacked on the first light-emitting layer, wherein the second light-emitting layer has a second peak emission wavelength which is different from the first peak emission wavelength.

According to an exemplary embodiment, the peak emission wavelength of the first light-emitting layer is in a range of 380 nm to 500 nm or 650 nm to 1000 nm.

According to an exemplary embodiment, the peak emission wavelength of the first light-emitting layer is in one of a range of 380 nm to 500 nm and a range of 650 nm to 1000 nm, and the peak emission wavelength of the second light-emitting layer is in the other of the range of 380 nm to 500 nm and the range of 650 nm to 1000 nm.

According to an exemplary embodiment, the method further comprises: disposing a light transmittable liner on a light exiting side of the flexible OLED device.

According to an exemplary embodiment, the light transmittable liner comprises at least one removable fabric layer.

According to an exemplary embodiment, the at least one removable fabric layer comprises a plurality of removable fabric layers which have meshes.

According to an exemplary embodiment, the method further comprises: disposing a light shielding layer on one side of the flexible OLED device opposite to the light exiting side to shield light emitted by the flexible OLED device to an external environment.

According to an exemplary embodiment, the wearable apparatus further comprises: a controller connected to the flexible OLED device to control an operation of the flexible OLED device; and a temperature sensor configured to detect a temperature on a light exiting side of the flexible OLED device, wherein the temperature sensor is connected to the controller so that the controller controls to turn off the flexible OLED device when the detected temperature is greater than a temperature threshold.

According to an exemplary embodiment, the wearable apparatus further comprises: a timer configured to count an on-time of the flexible OLED device so that the flexible OLED device is turned off when the counted time is greater than a time threshold

### BRIEF DESCRIPTION OF THE DRAWINGS

In order to more clearly illustrate the technical solutions according to the embodiments of the present disclosure, the accompanying drawings need to be used in the description of the embodiments will be briefly described below. Obviously, the accompanying drawings in the following description are only some embodiments of the present disclosure, and other accompanying drawings may be obtained by those of ordinary skill in the art according to these accompanying drawings without any creative work. In the accompanying drawings,

FIG. 1 illustrates a schematic block diagram of a wearable apparatus according to an exemplary embodiment of the present disclosure;

FIG. 2 illustrates an exemplary cross-sectional view of a flexible OLED device according to an exemplary embodiment of the present disclosure;

FIG. 3 illustrates an exemplary cross-sectional view of a flexible OLED device according to another exemplary embodiment of the present disclosure;

FIG. 4 illustrates a cross-sectional view of a portion of a wearable apparatus in which a flexible OLED device is disposed according to another exemplary embodiment of the present disclosure;

FIG. 5 illustrates an example of a light transmittable liner of FIG. 4;

FIG. 6 illustrates a cross-sectional view of a portion of a wearable apparatus in which a flexible OLED device is disposed according to another exemplary embodiment of the present disclosure;

FIG. 7 illustrates a schematic block diagram of a wearable apparatus according to another exemplary embodiment of the present disclosure;

FIG. 8 illustrates a schematic block diagram of a wearable apparatus according to another exemplary embodiment of the present disclosure;

FIG. 9 illustrates a schematic block diagram of a wearable device according to an exemplary embodiment of the present disclosure; and

FIG. 10 illustrates a flowchart of a method for manufacturing a wearable apparatus according to an exemplary embodiment of the present disclosure.

### DETAILED DESCRIPTION

In order to make the purposes, technical solutions and advantages of the embodiments of the present disclosure more clear, the technical solutions in the embodiments of the present disclosure will be clearly and completely described below in conjunction with the accompanying drawings in the embodiments of the present disclosure. Obviously, the embodiments described are a part of the embodiments of the present disclosure instead of all the embodiments. All other embodiments obtained by those of ordinary skill in the art based on the described embodiments of the present disclosure without contributing any creative work are within the protection scope of the present disclosure. It should be noted that throughout the accompanying drawings, the same elements are represented by the same or similar reference signs. In the following description, some specific embodiments are for illustrative purposes only and are not to be construed as limiting the present disclosure, but merely examples of the embodiments of the present disclosure. The conventional structure or construction will be omitted when it may cause confusion with the understanding of the present disclosure. It should be noted that shapes and dimensions of components in the figures do not reflect true sizes and proportions, but only illustrate contents of the embodiments of the present disclosure.

Unless otherwise defined, technical terms or scientific terms used in the embodiments of the present disclosure should be of ordinary meanings to those skilled in the art. “First”, “second” and similar words used in the embodiments of the present disclosure do not represent any order, quantity or importance, but are merely used to distinguish between different constituent parts.

Generally, for certain discomforts of a subject such as a human body, the discomforts of the subject may be alleviated or cured by illuminating the subject with light of different wavelength bands. For example, for neonatal pathological jaundice, a newborn suffering from the disease may be placed in a blue light illumination environment with a certain illumination, so that a skin of the newborn is directly illuminated with blue light of a specific wavelength until the neonatal jaundice subsides. Further, for a cervical vertebra problem, rheumatism, arthritis, etc. of a human body, a good therapeutic effect may be achieved by illuminating the human body with infrared rays or near infrared rays. However, the existing technical solutions have certain limitations. For example, an existing approach for treating neonatal jaundice is to provide a blue light treatment box in a blue light illumination environment. The blue light treatment box needs to shield portions of the newborn, for example, a head of the newborn etc., to protect the newborn. As a result, the process is complicated to operate, and the newborn has a poor comfort. In addition, for example, an existing approach for treating a cervical vertebrae problem is to provide an infrared treatment belt with an infrared light emitting apparatus which is attached to a neck of a subject. However, due to the limitations that the existing infrared light emitting apparatus is large in volume, the existing infrared treatment belt is too large and too bulky, thus resulting in a poor experience of the subject.

Since an OLED device has a series of characteristics, for example, self-luminous, free of backlight modules, adaption to a flexible panel, good temperature characteristics, low power consumption, high response speed, low manufacturing cost etc., the flexible OLED device is light and thin and has flexibility, and may be applied to a wearable apparatus, especially for treatment using light illumination in the field of health care.

According to the embodiments of the present disclosure, a flexible OLED device is used as an illumination unit, to realize a simple and effective health care function by illuminating a human body with light of different light-emitting bands.

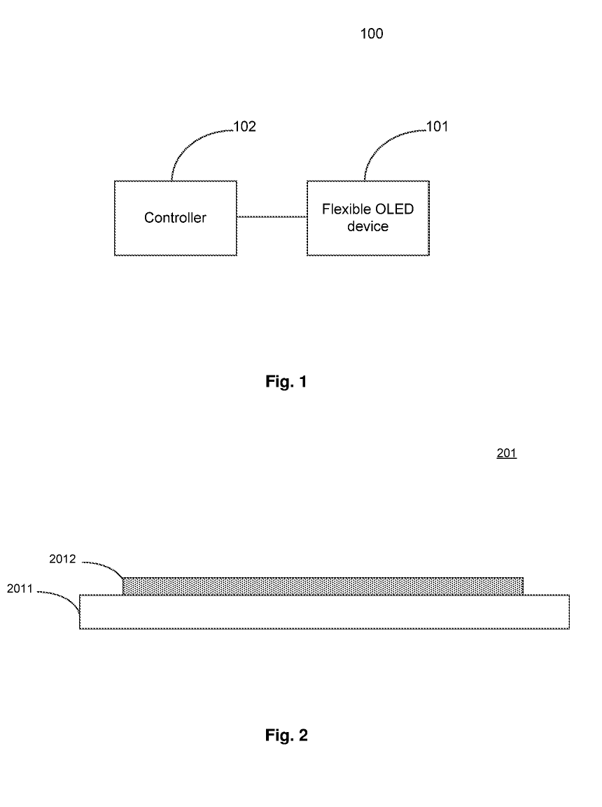

FIG. 1 illustrates a schematic block diagram of a wearable apparatus according to an embodiment of the present disclosure. As shown in FIG. 1, the wearable apparatus 100 according to an embodiment of the present disclosure may comprise a flexible OLED device 101 including a first light-emitting layer having a first peak emission wavelength; and a controller 102 connected to the flexible OLED device 101 to control an operation of the flexible OLED device 101.

FIG. 2 illustrates an exemplary cross-sectional view of a flexible OLED device according to an embodiment of the present disclosure. As shown in FIG. 2, the flexible OLED device 201 according to an embodiment of the present disclosure may comprise a substrate 2011 and a first light-emitting layer 2012, wherein the first light-emitting layer 2012 has a first peak emission wavelength.

It may be understood by those skilled in the art that the substrate 2011 may be a flexible substrate. For example, the flexible substrate may comprise paper, fabric, metal foil, a flexible glass layer and/or a polymer layer. The flexible substrate may be made transparent or opaque according to practical requirements. The flexible OLED device 201 may further comprise a structure including, for example, an anode, a hole injection layer, a hole transport layer, an electron blocking layer, a hole blocking layer, an electron transport layer, an electron injection layer, a cathode etc. For the sake of brevity, the known structures described above will not be described again herein.

According to an embodiment of the present disclosure, the peak emission wavelength of the first light-emitting layer 2012 may be in a range of 380 nm to 500 nm or 650 nm to 1000 nm. That is, the peak emission wavelength of the first light-emitting layer 2012 may be in a blue spectral band or in an infrared spectral band. For example, the first light-emitting layer 2012 may be implemented as an organic light-emitting layer including a base material and a dopant, such as a phosphorous dopant. It may be understood by those skilled in the art that the peak emission wavelength of the first light-emitting layer may be adjusted by adjusting a composition and a ratio of the dopant. For the sake of brevity, the known structures described above will not be described again herein.

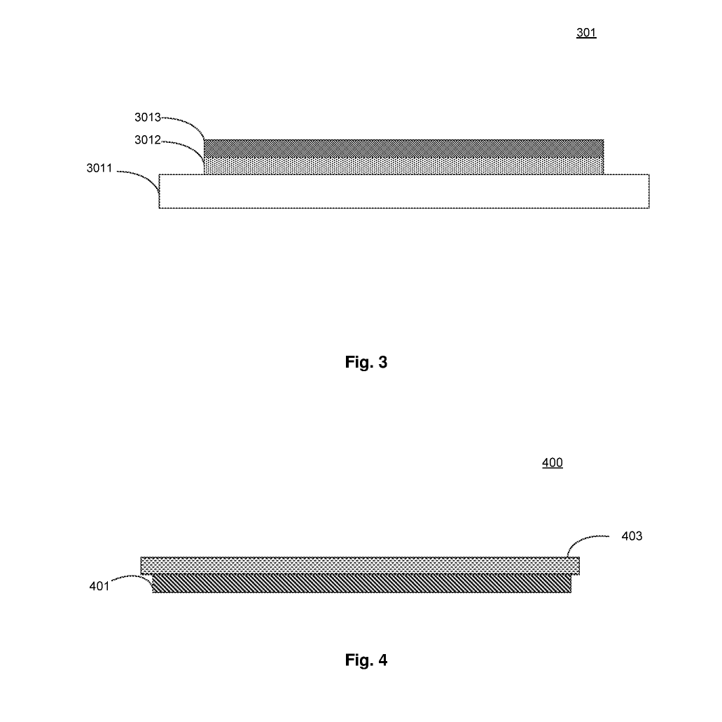

FIG. 3 illustrates an exemplary cross-sectional view of a flexible OLED device according to another exemplary embodiment of the present disclosure. As shown in FIG. 3, in addition to the substrate 3011 and the first light-emitting layer 3012, the flexible OLED device 301 according to another embodiment of the present disclosure further comprises a second light-emitting layer 3013 stacked on the first light-emitting layer 3012, wherein the second light-emitting layer 3013 has a second peak emission wavelength which is different from the first peak emission wavelength.

According to an embodiment of the present disclosure, the peak emission wavelength of the first light-emitting layer 3012 is in one of a range of 380 nm to 500 nm and a range of 650 nm to 1000 nm, and the peak emission wavelength of the second light-emitting layer 3013 is in the other of the range of 380 nm to 500 nm and the range of 650 nm to 1000 nm. That is, the peak emission wavelength of the first light-emitting layer 3012 may be in one of a blue spectral band or an infrared spectral band, and the peak emission wavelength of the second light-emitting layer 3013 may be in the other of the blue spectral band or the infrared spectral band. For example, the first light-emitting layer 3012 and the second light-emitting layer 3013 may be implemented as organic light-emitting layers each including a base material and a dopant such as a phosphorus dopant. It may be understood by those skilled in the art that each of the peak emission wavelengths of the first light-emitting layer 3012 and the second light-emitting layer 3013 may be adjusted by adjusting a composition and a ratio of a corresponding dopant. For the sake of brevity, the known structures described above will not be described again herein.

FIG. 4 illustrates a cross-sectional view of a portion of a wearable apparatus in which a flexible OLED device is disposed according to another exemplary embodiment of the present disclosure. As shown in FIG. 4, the wearable apparatus 400 according to another exemplary embodiment of the present disclosure may further comprise a light transmittable liner 403 disposed on a light exiting side of the flexible OLED device 401. It may be understood by those skilled in the art that the flexible OLED device 401 may have the structure described with reference to FIG. 2 or FIG. 3, which is not limited in the present disclosure. For example, the light transmittable liner 403 may have an area equal to or slightly greater than a light-emitting area of the flexible OLED device 401.

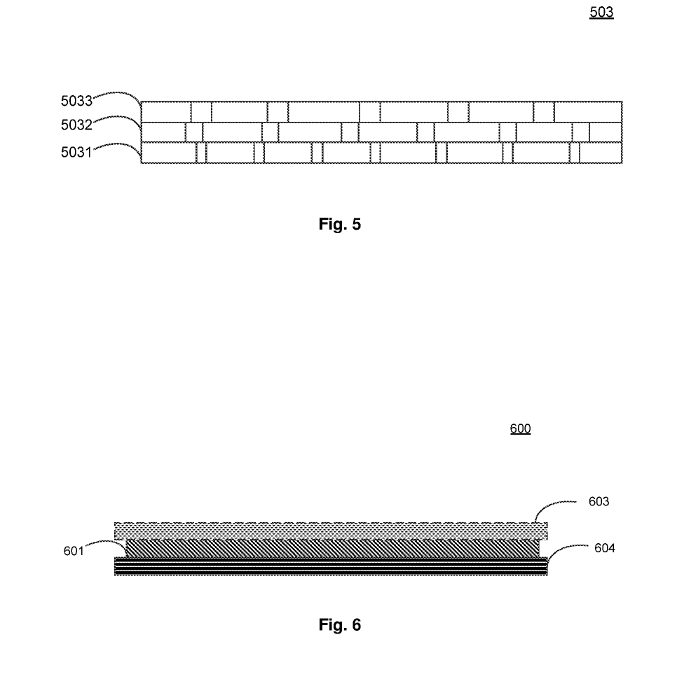

FIG. 5 illustrates an example of the light transmittable liner 403 of FIG. 4. As shown in FIG. 5, a light transmittable liner 503 may comprise at least one removable fabric layer. For example, the at least one removable fabric layer comprises a plurality of removable fabric layers 5031-5033, which may have meshes. The meshes may have different sizes or the same size, may have any shape, and may be evenly distributed or unevenly distributed, as long as the light-transmittable liner is disposed on the light exiting side of the OLED device so as to be able to properly shield light with excessive intensity and improve a comfort of a subject who uses the wearable apparatus. For example, the removable fabric may be realized as a light transmittable, heat resistant and skin-friendly material, for example, cotton, hemp etc.

FIG. 6 illustrates a cross-sectional view of a portion of a wearable apparatus in which a flexible OLED device is disposed according to another exemplary embodiment of the present disclosure. As shown in FIG. 6, the wearable apparatus 600 according to another exemplary embodiment of the present disclosure further comprises a light shielding layer 604 which covers the flexible OLED device 601 to shield light emitted by the flexible OLED device 601 to an external environment. The wearable apparatus 600 may further comprise a light transmittable liner 603 disposed on one side of the flexible OLED device 601 opposite to the light shielding layer 604. It may be understood by those skilled in the art that the flexible OLED device 601 may have the structure described with reference to FIG. 2 or FIG. 3, and the light transmittable liner 603 may have the structure shown in FIG. 5, which is not limited in the present disclosure. Herein, the term “external environment” refers to one side of the flexible OLED device 601 opposite to the light exiting side thereof.

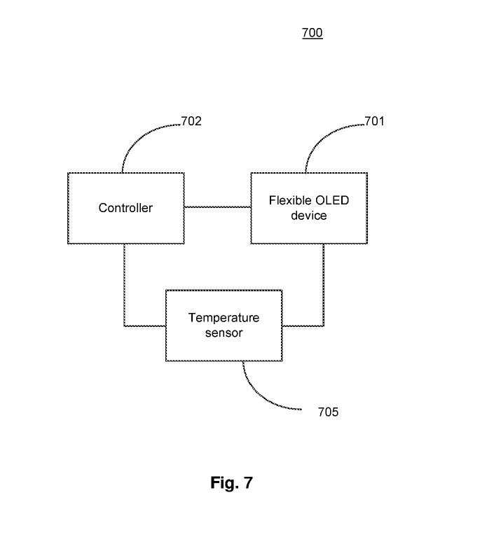

FIG. 7 illustrates a schematic block diagram of a wearable apparatus according to another exemplary embodiment of the present disclosure. As shown in FIG. 7, the wearable apparatus 700 according to another exemplary embodiment of the present disclosure may comprise a flexible OLED device 701; a controller 702; and a temperature sensor 705 configured to detect a temperature on a light exiting side of the flexible OLED device 701. The temperature sensor 705 is connected to the controller 702, so that the controller 702 controls to turn off the flexible OLED device 701 when the detected temperature is greater than a temperature threshold. The temperature threshold may be set in a range of 25 degrees Celsius to 30 degrees Celsius, and when the detected temperature is greater than the temperature threshold, the controller 702 controls to turn off the flexible OLED device 601 to prevent overheating, thereby further improving the safety of the wearable apparatus.

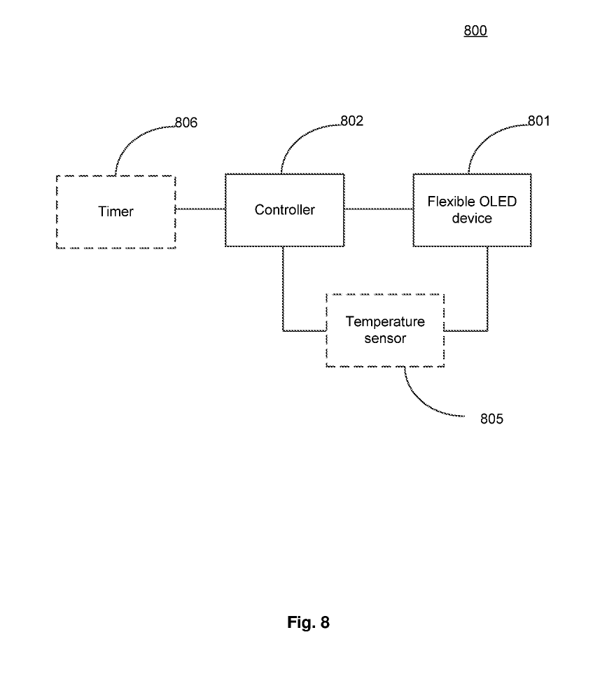

FIG. 8 illustrates a schematic block diagram of a wearable apparatus according to another exemplary embodiment of the present disclosure. As shown in FIG. 8, the wearable apparatus 800 according to another exemplary embodiment of the present disclosure may comprise a flexible OLED device 801 and a controller 802. The wearable apparatus 800 may further comprise a timer 806 configured to count turn-on time of the flexible OLED device 801, so that the flexible OLED device 801 is turned off when the counted turn-on time is greater than a time threshold. The time threshold may be set according to practical requirements, thereby further improving the convenience in usage of the wearable apparatus. It may be understood by those skilled in the art that the timer 806 may be a discrete device, or the timer 806 may be integrated into the controller 802 to implement a function of the timer 806, which is not limited in the present disclosure. According to another exemplary embodiment, the wearable apparatus 800 may further comprise a temperature sensor 805.

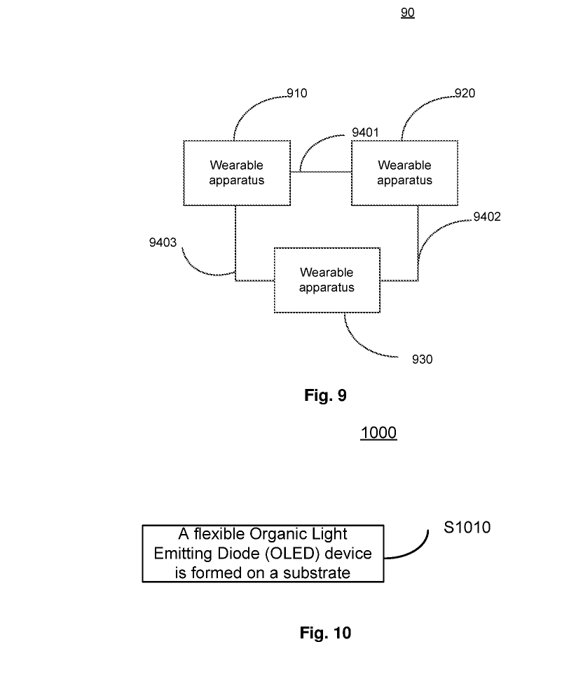

The embodiments of the present disclosure further provide a wearable device including at least one wearable apparatus according to the embodiments of the present disclosure as described above. FIG. 9 illustrates a schematic block diagram of a wearable device 90 according to an embodiment of the present disclosure. As shown in FIG. 9, the wearable device 90 according to an embodiment of the present disclosure may comprise: at least one wearable apparatuses 910-930 according to the embodiments of the present disclosure. Although FIG. 9 illustrates that the wearable device 90 comprises three wearable apparatuses 910-930 by way of example, it may be understood by those skilled in the art that the wearable device 90 may comprise any number of wearable apparatuses according to practical requirements, for example, in a case of a subject with neonatal jaundice, the wearable device may comprise six wearable apparatuses which cover a front chest, a back, and extremities of the subject, respectively.

As shown in FIG. 9, at least some of the plurality of wearable apparatuses 910-930 may be connected to each another via length adjustable mechanisms 9401-9403, so that distances between the wearable apparatuses may be adjusted according to statures of different subjects.

Further, although FIG. 9 illustrates that the plurality of wearable apparatuses may be connected to each other via the length adjustable mechanisms, some of the plurality of wearable apparatuses may be connected via length adjustable mechanisms. The plurality of wearable apparatuses may also be independent of each other. For example, for a subject suffering from a joint disease, a plurality of wearable apparatuses according to the embodiments of the present disclosure may be worn separately on joints of the subject without affecting freedom of actions of the subject. A specific connection manner may be set by those skilled in the art according to practical requirements.

FIG. 10 illustrates a flowchart of a method for manufacturing a wearable apparatus according to an exemplary embodiment of the present disclosure. As shown in FIG. 10, the method 1000 for manufacturing a wearable apparatus according to the embodiment of the present disclosure may comprise the following steps.

In step 1010, a flexible OLED device is formed on a substrate. The flexible OLED device comprises a first light-emitting layer having a first peak emission wavelength.

In an exemplary embodiment, the peak emission wavelength of the first light-emitting layer is in a range of 380 nm to 500 nm or 650 nm to 1000 nm. The flexible OLED device further comprises a second light-emitting layer stacked on the first light-emitting layer, wherein the second light-emitting layer has a second peak emission wavelength which is different from the first peak emission wavelength. The peak emission wavelength of the first light-emitting layer is in one of a range of 380 nm to 500 nm and a range of 650 nm to 1000 nm, and the peak emission wavelength of the second light-emitting layer is in the other of the range of 380 nm to 500 nm and the range of 650 nm to 1000 nm.

In an exemplary embodiment, the method for manufacturing a wearable apparatus further comprises a step of disposing a light transmittable liner on a light exiting side of the flexible OLED device. The light transmittable liner comprises at least one removable fabric layer. The at least one removable fabric layer comprises a plurality of removable fabric layers which have meshes.

In an exemplary embodiment, the method for manufacturing a wearable apparatus further comprises a step of disposing a light shielding layer on the other side of the flexible OLED device opposite to the light exiting side to shield light emitted by the flexible OLED device to an external environment.

According to the embodiments of the present disclosure, the flexible OLED device is used as an illumination unit, to realize a simple and effective health care function by illuminating a human body with light of different light-emitting bands.

Although the present invention has been particularly shown and described with reference to the exemplary embodiments of the present disclosure, it should be understood by those skilled in the art that many changes may be made to these embodiments in form and details without departing from the spirit and scope of the present invention as defined by the appended claims.

1. A wearable apparatus, comprising:
a flexible Organic Light Emitting Diode (OLED) device comprising a first light-emitting layer having a first peak emission wavelength and a second light-emitting layer stacked on the first light-emitting layer, wherein the second light-emitting layer has a second peak emission wavelength which is different from the first peak emission wavelength; and
a controller connected to the flexible OLED device to control an operation of the flexible OLED device.

2. The wearable apparatus according to claim 1, wherein the peak emission wavelength of the first light-emitting layer is in a range of 380 nm to 500 nm or 650 nm to 1000 nm.

3. (canceled)

4. The wearable apparatus according to claim 1, wherein the peak emission wavelength of the first light-emitting layer is in one of a range of 380 nm to 500 nm and a range of 650 nm to 1000 nm, and the peak emission wavelength of the second light-emitting layer is in the other of the range of 380 nm to 500 nm and the range of 650 nm to 1000 nm.

5. The wearable apparatus according to claim 1, further comprising:
a light transmittable liner disposed on a light exiting side of the flexible OLED device.

6. The wearable apparatus according to claim 5, wherein the light transmittable liner comprises at least one removable fabric layer.

7. The wearable apparatus according to claim 6, wherein the at least one removable fabric layer comprises a plurality of removable fabric layers which have meshes.

8. The wearable apparatus according to claim 5, further comprising:
a light shielding layer which covers one side of the flexible OLED device opposite to the light exiting side to shield light emitted by the flexible OLED device to an external environment.

9. The wearable apparatus according to claim 1, further comprising:
a temperature sensor configured to detect a temperature on a light exiting side of the flexible OLED device,
wherein the temperature sensor is connected to the controller so that the controller controls to turn off the flexible OLED device when the detected temperature is greater than a temperature threshold.

10. The wearable apparatus according to claim 1, further comprising:
a timer configured to count turn-on time of the flexible OLED device so that the flexible OLED device is turned off when the counted turn-on time is greater than a time threshold.

11. A wearable device, comprising at least one wearable apparatus according to claim 1.

12. The wearable device according to claim 11, wherein the at least one wearable apparatus comprises a plurality of wearable apparatuses, wherein at least some of the plurality of wearable apparatuses are connected to each other via length adjustable mechanisms.

13. A method for manufacturing a wearable apparatus, comprising:
forming a flexible Organic Light Emitting Diode (OLED) device on a substrate, wherein the flexible OLED device comprises a first light-emitting layer having a first peak emission wavelength and a second light-emitting layer stacked on the first light-emitting layer,
wherein the second light-emitting layer has a second peak emission wavelength which is different form the first peak emission wavelength.

14. The method according to claim 13, wherein the peak emission wavelength of the first light-emitting layer is in a range of 380 nm to 500 nm or 650 nm to 1000 nm.

15. (canceled)

16. The method according to claim 13, wherein the peak emission wavelength of the first light-emitting layer is in one of a range of 380 nm to 500 nm and a range of 650 nm to 1000 nm, and the peak emission wavelength of the second light-emitting layer is in the other of the range of 380 nm to 500 nm and the range of 650 nm to 1000 nm.

17. The method according to claim 13, further comprising:
disposing a light transmittable liner on a light exiting side of the flexible OLED device.

18. The method according to claim 17, wherein the light transmittable liner comprises at least one removable fabric layer.

19. The method according to claim 18, wherein the at least one removable fabric layer comprises a plurality of removable fabric layers which have meshes.

20. The method according to claim 17, further comprising:
disposing a light shielding layer on one side of the flexible OLED device opposite to the light exiting side to shield light emitted by the flexible OLED device to an external environment.

21. The method according to claim 13, wherein the wearable apparatus further comprises:
a controller connected to the flexible OLED device to control an operation of the flexible OLED device; and
a temperature sensor configured to detect a temperature on a light exiting side of the flexible OLED device,
wherein the temperature sensor is connected to the controller so that the controller controls to turn off the flexible OLED device when the detected temperature is greater than a temperature threshold.

22. The method according to claim 13, wherein the wearable apparatus further comprises:
a timer configured to count an on-time of the flexible OLED device so that the flexible OLED device is turned off when the counted time is greater than a time threshold.
